(19) United States
(12) Patent Application Publication
(10) Pub. No.: US 2023/0184751 A1
(43) Pub. Date: Jun. 15, 2023

(54) FLOW CHEMISTRY SYSTEM AND METHOD FOR CARBOHYDRATE ANALYSIS

(71) Applicants: Ming-Che SHIH, Culver City, CA (US); ACADEMIA SINICA, Taipei City (TW)

(72) Inventor: Wen-Bin YANG, Taipei City (TW)

(73) Assignee: ACADEMIA SINICA, Taipei City (TW)

(21) Appl. No.: 17/925,119

(22) PCT Filed: May 14, 2021
(86) PCT No.: PCT/US2021/032420

§ 371 Date: Nov. 14, 2022

Related U.S. Application Data
(60) Provisional application No. 63/025,184, filed on May 15, 2020.

(51) Int. Cl. G01N 33/53; G01N 30/72; G01N 33/58

(52) U.S. Cl. CPC G01N 33/5308; G01N 30/7233; G01N 33/58; G01N 2030/027

(57) ABSTRACT

The present invention relates to a system and method for carbohydrate analysis in a flow chemistry manner. The present invention at least features continuous reactions of glycan hydrolysis in combination with saccharide labeling which is helpful to improve the existing approaches in glycan structural analysis.

### RELATED APPLICATIONS

This application claims the benefit of U.S. provisional application No. 63/025,184, filed May 15, 2020 under 35 U.S.C. § 119, the entire content of which is incorporated herein by reference.

### TECHNOLOGY FIELD

The present invention relates to a system and method for carbohydrate analysis in a flow chemistry manner. The present invention at least features continuous reaction of glycan hydrolysis and saccharide labeling which is helpful to improve the existing approaches in glycan structural analysis.

### BACKGROUND

Carbohydrate analysis is essential for using glycans in biological research, clinical analysis, and biotechnological production.¹ The primary structure of a glycan is defined not only by the constituent monosaccharides, but also by their linkages and branching. Often the nature and position of nonglycan substituents such as aglycan, esters (e.g., acetate, sulphate and phosphate) need to be determined. Finally, methods for solving the three-dimensional structures of glycans are also needed.

Various approaches for analyses of glycan structure have been reported.²⁻⁵ Structural and compositional analysis of glycans often require hydrolysis to release the monosaccharides, for which acidic hydrolysis is most typically used. Many hydrolysis protocols for glycan hydrolysis have been reported.⁶⁻¹¹ Monosaccharide analyses are usually by liquid chromatography (LC), mass spectrometry (MS), nuclear magnetic resonance (NMR) or any combination of the three techniques. Moreover, the released monosaccharides can be derivatized to facilitate the detection and quantification by LC analysis.^{12,13} Suitable derivatization also aids in improving ionization efficiency for MS analysis. Capillary electrophoresis mass spectrometry (CE-MS),¹⁴⁻¹⁶ LC-MS¹⁷⁻¹⁹ and NMR^{20,21} can be used to determine the structures of complex glycans and substantial to extend over NADA tagging.²² We have previously explored a method using naphthalene-2,3-diamine (NADA) for derivatization of aldoses and α-ketoacid type saccharides (e.g., sialic acid) to their corresponding naphthimidazole (NAIM) and quinoxalinone (QXO) derivatives (FIG. 1).²²⁻²⁴ Conversion of aldoses by reductive amination at the reducing terminals is a common practice to afford the derivatives for mass spectrometric analyses.²⁴ However, the high content of salts in the products needs to be removed to increase the signal level. Conjugating α-ketoacid by reductive amination is not effective due to low reactivity and yield of two isomers. In comparison, the NAIM and QXO sugars are readily prepared to assist the structural assignment of parent sugars in the chromatographic and spectrometric analyses.²²⁻²⁶

A reducing sugar may exist in the cyclic form as the α and β anomers, which sometimes obscure ¹H-NMR signals. The sugar-NAIM derivative eliminates this obstacle in NMR analysis.²⁵ We have previously shown that NAIM derivatization provides a simple method for quantitative NMR analysis of monosaccharides and disaccharides, including arabinose (Ara), xylose (Xyl), rhamnose (Rha), glucose (Glc), mannose (Man), galactose (Gal), N-acetylgalactosamine (GalNAc), glucuronic acid (GlcUA), maltose (Mal) and lactose (Lac).²⁵ The NAIM derivative of each saccharide shows a single characteristic vinyl H-2 proton at a distinct position to facilitate the quantitative analysis. This NAIM method is especially useful for identification and quantification of multiple kinds of glycans for their compositional analysis. In addition, the sugar-NAIM carries a hydrophobic naphthimidazole group that can enhance ionization in MS detection.²⁶ The UV and fluorescence active NAIM modifier can also assist in the LC analysis. The limit of detection for sugar-NAIM compounds can possibly reach sub-micromolar range on using a fluorescence detector. Furthermore, the D-/L-enantiomeric pairs of sugar-NAIM compounds derived from common monosaccharides, including ribose (Rib), Ara, Xyl, Rha, fucose (Fuc), Glc, Man, Gal, N-acetylgalactosamine (GalNAc), GlcUA and galacturonic acid (GalUA), are resolved on an uncoated fused-silica capillary using sulfated-α-cyclodextrin as the chiral selector.²⁷

### SUMMARY

The present invention provides a new technology for carbohydrate analysis in a flow chemistry manner. In particular, the present invention features combining glycan degradation and saccharide derivatization in a flow system, optionally along with the use of detection means, for example, chromatography, MS and NMR techniques, leading to rapid carbohydrate compositional analysis.

In one aspect, the present invention provides a method for analyzing a glycan molecule, comprising the steps of:
(i) degrading the glycan molecule in a hydrolysis reaction to produce a glycan hydrolysate containing monosaccharides;
(ii) labeling the monosaccharides with a detectable (e.g. fluorescent) label in a sugar derivatization reaction to produce sugar derivatives;
(iii) analyzing the sugar derivatives for measurement of one or more characteristics of the sugar derivatives; and
(iv) determining the composition and/or structure of the glycan molecule based on the one or more characteristics of the sugar derivatives,
wherein the glycan hydrolysis reaction of step (i) and the sugar derivation reaction of
step (ii) are performed in a flow chemistry system.

In another aspect, the present invention provides a flow chemistry system for analyzing a glycan molecule, comprising
(i) a hydrolysis unit for performing a hydrolysis reaction to degrade the glycan molecule to produce a glycan hydrolysate containing monosaccharides; and
(ii) a derivatization unit for performing a sugar derivatization reaction to label the monosaccharides with a detectable label to produce sugar derivatives,
wherein the hydrolysis unit is connected to the derivatization unit via connective tubing to provide a continuous flow path where the glycan hydrolysate flows from the hydrolysis unit into the derivatization unit for the sugar derivatization reaction.

In a further aspect, the prevent invention provides an apparatus for analyzing a glycan molecule, which comprises
(a) a flow chemistry system, comprising
   (i) a hydrolysis unit for performing a hydrolysis reaction to degrade a glycan molecule to produce a glycan hydrolysate containing monosaccharides; and
   (ii) a derivatization unit for performing a sugar derivatization reaction to label the monosaccharides with a detectable label to produce sugar derivatives,
   wherein the hydrolysis unit is connected to the derivatization unit via connective tubing to provide a continuous flow path where the glycan hydrolysate flows from the hydrolysis unit into the derivatization unit for the sugar derivatization reaction;
(b) an analytical system adapted for interaction with the flow chemistry system for the measurement of one or more characteristics of the sugar derivatives;
(c) a data processing system comprising sugar database and a means for comparing the one or more characteristics of the sugar derivatives measured by the analytical system with the sugar database to determine the composition and sugar sequence of the glycan molecule.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

FIG. 1 shows derivatizing glucose and sialic acid with naphalene-2,3-diamine (NADA).

FIG. 2 shows the diagram for preparation of sugar-NAIM derivatives in a flow chemistry system in certain embodiments of the present invention. Glacial acetic acid is shown in aqua blue. BPR is back pressure regulator.

FIG. 3 shows the diagram for glycan hydrolysis in a flow chemistry system. The gauge pressure of BPR was set as 3 and 4 bar for the reaction at 120° C. and 150° C., respectively.

FIG. 4 shows Glc-NAIM formation at different times and temperatures in a flow chemistry system. (A) 5 min at 25° C., (B) 10 min at 25° C., (C) 20 min at 25° C., and (D) 10 min at 60° C. The reaction was monitored by ¹H-NMR spectra (600 MHz, D₂O). The α- and β-anomers of glucose showed anomeric H signals at δ 5.22 and 4.64, respectively. The Glc-NAIM derivative showed the characteristic C-2 and C-3 protons at δ 5.38 and 4.39.

FIG. 5 shows formation of various sugar-NAIM derivatives at 60° C. for 10 min in a flow chemistry system. The final amounts of monosaccharide (15.0 mg, 0.08 mmol), NADA (30.0 mg, 0.18 mmol) and iodine (3.8 mg, 0.03 mmol) were used in each run of NAIM tagging reaction. ¹H-NMR spectra (600 MHz, D2O) were measured to characterize the sugar-NAIM derivatives by their C-2 protons: Glc-NAIM at δ 5.38, Gal-NAIM at δ 5.54, GlcUA-NAIM at δ 5.39, Fuc-NAIM at δ 5.61, Man-NAIM at δ 5.18, and Xyl-NAIM at δ 5.27.

FIG. 6 shows hydrolysis of maltotetraose (1.0 mg/mL) on treatment with 4 M HCl at 120° C. for different periods of time (10, 15 and 20 min) in a flow chemistry system. The reaction was monitored by MALDI-TOF-MS measurement.

FIG. 7 shows hydrolysis of SA-Gal-Glc trisaccharide (GM3-sugar, 1.0 mg/mL) with 4 M HCl at 120° C. for 10 min in a flow chemistry system. The reaction was monitored by MALDI-TOF-MS measurement.

FIG. 8 shows LC-MS analysis of the GM3-sugar hydrolysate after tagging with NADA to the corresponding NAIM and QXO derivatives. The reaction was monitored by LC-MS analysis: (A) LC diagram on a C18 capillary column, and (B) LTQ-FTMS spectra.

FIG. 9 shows the protocol of a continuous tandem strategy for glycan hydrolysis toward sugar-NAIM (or sugar-QXO) derivatives in a flow chemistry system in certain embodiments of the present invention.

FIG. 10 shows the protocol for glycan structural analysis by preparation of glycan beads for enzymatic cleavage and labeling the released saccharides in a flow chemistry system in certain embodiments of the present invention. DAB is 3,4-diaminobenzoic acid.

FIG. 11 shows the hydrolysis efficiency of maltose on treatment with 4 M HCl for 10 min at different temperatures in a flow chemistry system.

FIG. 12 shows the hydrolysis efficiency of maltotriose on treatment with 4 M HCl for 10 min at different temperatures in a flow chemistry system.

FIG. 13 shows comparison of the hydrolysis efficiency of maltotriose on treatment with 4 M or 2 M HCl at 120° C. for 10 min in a flow chemistry system.

FIG. 14 shows the hydrolysis efficiency of lactose with 4 M HCl at 120° C. for 10 min in a flow chemistry system.

FIG. 15 shows the ¹H-NMR spectrum of Lac-NAIM.

FIGS. 16A-16C shows the CE analysis of enzymatic digestion of oligosaccharides, including FIG. 16A, maltohexaose-NAIM derivative digested by α-amylase; FIG. 16B, laminarihexaose-NAIM derivative digested by endo-β-1,3-glucanase; and FIG. 16C, cellohexaose-NAIM derivative digested by cellulase.

### DETAILED DESCRIPTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The term “comprise” or “comprising” is generally used in the sense of include/including which means permitting the presence of one or more features, ingredients or components. The term “comprise” or “comprising” encompasses the term “consists” or “consisting of.”

As used herein, “around”, “about” or “approximately” can generally mean within 20 percent, particularly within 10 percent, and more particularly within 5 percent of a given value or range. Numerical quantities given herein are approximate, meaning that the term “around”, “about” or “approximately” can be inferred if not expressly indicated.

As used herein, the term “glycan” refers to an oligosaccharide or a polysaccharide. An oligosaccharide is a saccharide polymer composed of a relatively small number (e.g. two to ten) of monosaccharides, while a polysaccharide is a saccharide polymer composed of a relatively large number (e.g. more than ten) of monosaccharides. The term “monosaccharides” as used herein refers to the simplest form of carbohydrates. Examples of monosaccharides include but are not limited to glucose, fructose, galactose, xylose, mannose, fucose, rhamnose and ribose.

As used herein, the term “hydrolysis” with respect to a carbohydrate refers to the breakdown of a glycan molecule to monosaccharides, partially or completely. It can be carried out by an acidic hydrolysis process or enzymatic hydrolysis process.

As used herein, the term “sugar derivatization reaction” refers to a reaction to modify sugars for the purpose of their structural or functional analysis. Typically, most of the labels used for the derivatization of carbohydrates possess a chromophor or a flurophor which provides a sensitive detection of these analytes by means of spectroscopic methods, for example. In particular, a method using naphthalene-2,3-diamine (NADA) has been reported for derivatization of saccharides to their corresponding naphthimidazole (NAIM) derivatives. The NAIM derivative of a saccharide shows a single characteristic vinyl H-2 proton at a distinct position to facilitate the quantitative analysis and useful for identification and quantification of multiple kinds of glycans for their compositional analysis. Further, the sugar-NAIM derivatives carry a hydrophobic naphthimidazole group that can enhance ionization in MS detection, and also the UV and fluorescence active NAIM modifier can also assist in the LC analysis.

As used herein, the term “flow chemistry” refers to a process where a chemical reaction is run in a continuously flowing stream rather than in batch production. Typically, pumps move fluid into a tube, and where tubes join one another, the fluids contact one another. The use of a microreactor greatly facilitates the NAIM derivatization, resulting in a shorter reaction time and improved yield.²⁸ Flow chemistry systems for multiple-step synthesis of many other bioactive compounds and natural products²⁹⁻³² enhance the yield as well as contribute to safety.

According to the present invention, a new technology for carbohydrate analysis is provided which features combining the reactions of glycan hydrolysis and saccharide derivatization in a flow chemistry, leading to improvement of carbohydrate analysis.

In particular, the present invention provides a method for carbohydrate analysis, which comprises the steps of:
(i) degrading a glycan molecule in a hydrolysis reaction to produce a glycan hydrolysate containing monosaccharides;
(ii) labeling the monosaccharides with a detectable (e.g. fluorescent) label in a sugar derivatization reaction to produce sugar derivatives;
(iii) analyzing the sugar derivatives for measurement of one or more characteristics of the sugar derivatives; and
(iv) determining the composition and/or structure of the glycan molecule based on the one or more characteristics of the sugar derivatives,
wherein the glycan hydrolysis reaction of step (i) and the sugar derivation reaction of step (ii) are performed in a flow chemistry system.

In certain embodiments, the glucan molecule to be analyzed comprises oligosaccharides (e.g. di, tri, tetra saccharides) and/or polysaccharides.

In certain embodiments, the detectable label for the sugar derivation reaction is a naphthimidazole molecule.

In certain embodiments, the sugar derivatives are analyzed by nuclear magnetic resonance spectroscopy (NMR), liquid chromatography (LC), gas chromatography (GC), mass spectrometry (MS) and any combination thereof.

In certain embodiments, the glycan hydrolysis reaction is performed in a hydrolysis unit, the sugar derivation reaction is performed in a derivatization unit, and the hydrolysis unit is connected to the derivatization unit via connective tubing to provide a continuous flow path where the glycan hydrolysate flows from the hydrolysis unit into the derivatization unit for the sugar derivatization reaction.

In certain embodiments, the monosaccharides include ribose (Rib), arabinose (Ara), xylose (Xyl), rhamnose (Rha), fucose (Fuc), glucose (Glc), mannose (Man), galactose (Gal), N-acetylgalactosamine (GalNAc), glucuronic acid (GlcUA), and/or galacturonic acid (GalUA).

In certain embodiments, the hydrolysis reaction is effected by acidic hydrolysis or enzymatic hydrolysis.

In certain embodiments, the acidic hydrolysis is carried out under pH 1-5 at a temperature in the range of 60° C. to 150° C. for 5 to 120 minutes.

In certain embodiments, the enzymatic hydrolysis is performed with one or more enzymes selected from the group consisting of amylase, glucanase, cellulase, galactosidase, neuraminidase, glycosyltransferase, sialyltransferase, and any combinations thereof.

The present invention also provides a flow chemistry system for carbohydrate analysis, which comprises
(i) a hydrolysis unit for performing a hydrolysis reaction to degrade a glycan molecule to produce a glycan hydrolysate containing monosaccharides; and
(ii) a derivatization unit for performing a sugar derivatization reaction to label the monosaccharides with a detectable label to produce sugar derivatives; and
wherein the hydrolysis unit is connected to the derivatization unit via connective tubing to provide a continuous flow path where the glycan hydrolysate flows from the hydrolysis unit into the derivatization unit for the sugar derivatization reaction.

The present invention further provides an apparatus for performing a method for carbohydrate analysis as described herein. Specifically, the apparatus of the present invention comprises a flow chemistry system as described herein in combination with an analytical system adapted for interaction with the flow chemistry system for the measurement of one or more characteristics of the sugar derivatives, and a data processing system comprising sugar database and a means for comparing the one or more characteristics of the sugar derivatives measured by the analytical system with the sugar database to determine the composition and sugar sequence of the glycan molecule.

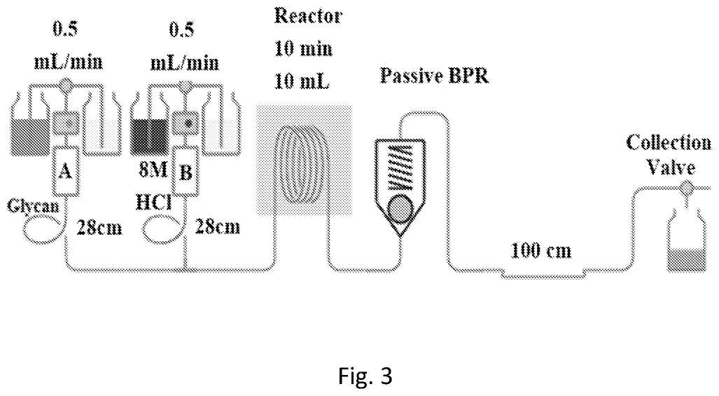

In certain embodiments, the hydrolysis unit includes a first reservoir A containing a solution of the glycan molecule, a first reservoir B containing an acidic solution, a hydrolysis reactor and a first collection valve, connected with connective tubing and configured to enable the solution of the glycan molecule and the acidic solution to flow into the hydrolysis reactor where the hydrolysis reaction is performed and the resultant glycan hydrolysate flows into the derivatization unit when the first collection valve is in an open position. See FIG. 3, for example.

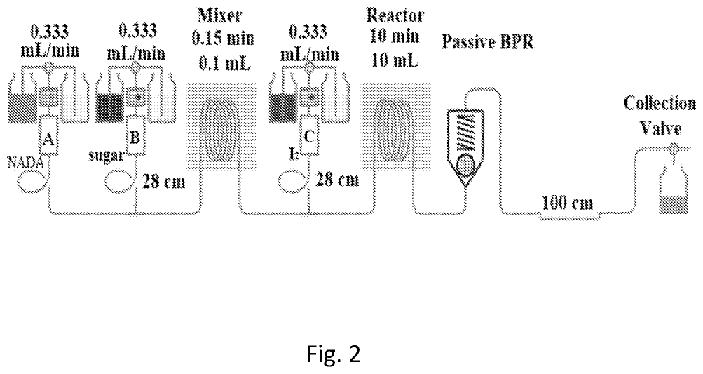

In certain embodiments, the derivatization unit includes a second reservoir A containing the fluorescent label, a second reservoir B containing the glycan hydrolysate, a mixer, a derivatization reactor and a second collection valve, connected with connective tubing and configured to enable the fluorescent label and the glycan hydrolysate to flow into the mixer to form a mixture of the fluorescent label and the glycan hydrolysate, and the mixture to flow into the derivatization reactor to produce the fluorescent labelled sugar derivatives. See FIG. 2, for example.

In certain embodiments, the measurement for one or more characteristics of the sugar derivatives is performed by nuclear magnetic resonance spectroscopy (NMR), liquid chromatography (LC), gas chromatography (GC), mass spectrometry (MS) and any combination thereof.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

This study demonstrates the utilization of flow chemistry system for continuous glycan hydrolysis and saccharide labeling to assist with the existing methods in glycan structural analysis. Acidic hydrolysis of glycans could be accelerated in a flow system. Aldoses and α-ketoacid type saccharides were effectively labeled with naphthalene-2,3-diamine (NADA) at 60° C. for 10 min to form the fluorescent naphthimidazole (NAIM) and quinoxalinone (QXO) derivatives, respectively. The NADA-labeled derivatives improved the structural determination and composition analysis for their parent saccharides by using matrix-assisted laser desorption ionization time-of-flight mass spectrometer (MALDI-TOF-MS), liquid chromatography mass spectrometer (LC-MS) and nuclear magnetic resonance (NMR). Furthermore, this protocol was applied to determine the SA-Gal-Glc sequence of GM3-sugar out of 6 possible permutations.

### 1. Material and Methods

### 1.1. Chemicals

Iodine, glacial acetic acid, NADA, HCl, and D₂O were purchased from Merck & Co., Inc. (Darmstadt, Germany). 2,5-Dihydroxybenzoic acid (2,5-DHB), glucose, maltose, maltotriose, lactose and other monosaccharides were purchased from Sigma-Aldrich (St. Louis, Miss., US). GM3-sugar was purchased from Dextra Laboratories Ltd. (Reading, UK). Maltotetraose was purchased from Supelco Analytical (Mainz, Germany). All chemicals and solvents were of analytical grade and used without further purification. The NAIM labeling kit used in this study was a gift from Sugarlighter Co., Inc. (New Taipei City, Taiwan).²⁵

### 1.2 Batch Preparation of Sugar-NAIM Derivatives

The procedure follows the published method.²² A mixture of monosaccharide (2.0 mg, 11 μmol), naphthalene-2,3-diamine (2.0 mg, 13 μmol) and iodine (2.0 mg, 8 μmol) in glacial acetic acid (1.0 mL) was stirred at room temperature. The labeling reaction was completed in 3 h as indicated by thin-layer chromatography (TLC). The mixture was concentrated by rotary evaporation under reduced pressure to give the sugar-NAIM derivative. Other sugars were also derivatized in this fashion. Alternatively, sugar-NAIM derivatives were prepared by using a NAIM labeling kit (Sugarlighter Co.).²⁵

### 1.3 Vapourtec E-series Flow Chemistry System

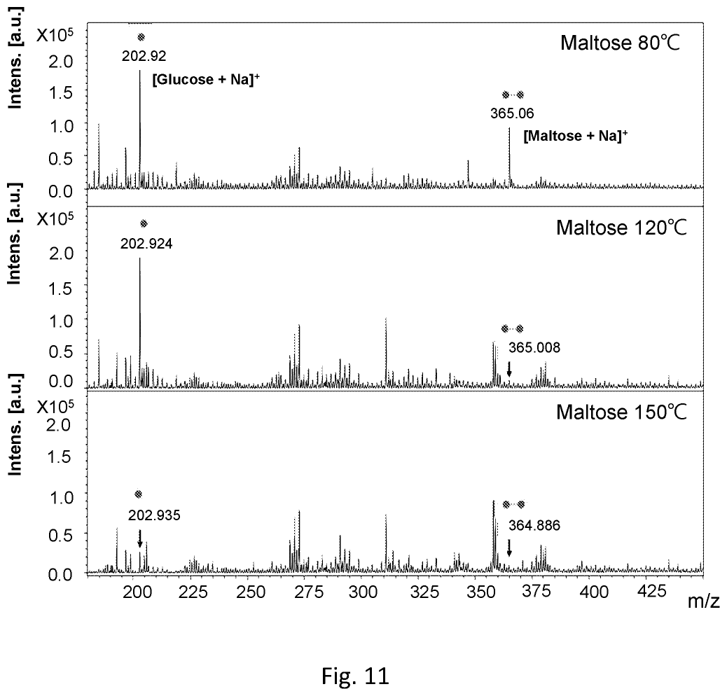

A Vapourtec flow reactor E-series with V-3 peristaltic pumps (Vapourtec Ltd., Bury St Edmunds, Suffolk, UK) was used for flow chemistry. Our setup is shown in FIG. 11. The reactor comprises a 10.0 mL 1/16″ PTFE tube (0.81 mm i.d.×200 cm). The E-series comes with a touchscreen interface, mounted at an ergonomically optimal height with full tilt adjustment. It allows setting the key flow rates and the temperature (±1° C.) through a feedback system.

### 1.4 Method for Preparation of Sugar-NAIM Derivatives in Flow Chemistry System

The procedure for preparing sugar-NAIM in a flow chemistry system was modified from the batch preparation method.^{22,25} The flow-diagram of NAIM tagging process in Vapourtec easy-MedChem flow chemistry system is shown in FIG. 2. All solutions were in glacial HOAc: vial A, naphthalene-2,3-diamine (NADA, 1000 mg/100 mL); vial B, sugar samples (500 mg/100 mL); and vial C, iodine (127 mg/100 mL). The reaction was performed by pumping the A, B and C solutions at the same rate (0.33 mL/min). The final amount of monosaccharide (15.0 mg, 0.08 mmol), NADA (30.0 mg, 0.18 mmol), and iodine (3.8 mg, 0.03 mmol) was conducted at 60° C. (the reading of instrument setting) over a period of 10 min. After the reaction was completed, the mixture was concentrated by rotary evaporation under reduced pressure to give the desired sugar-NAIM derivative, which was directly subjected to ¹H-NMR and LC-MS analyses without further purification. This reaction protocol is applicable to prepare other sugar-NAIM derivatives, including those of mixed sugars, oligosaccharides and glycans.

### 1.5 Procedure for Glycan Hydrolysis in Flow Chemistry System

The diagram of glycan hydrolysis set-up in a flow chemistry system is shown in FIG. 3. Vial A containing a solution of glycan (100 mg) in doubly distilled water (dd-H₂O, 100 mL) and vial B containing a solution of 8 M HCl (100 mL) were prepared for glycan hydrolysis. The reaction was performed by pumping A and B solutions at the same rate (0.5 mL/min) at various temperatures over a period of 10 min. This generated a hydrolysis volume of 10.0 mL with a concentration of 5.0 mg of glycan in 4 M HCl. After the reaction was completed, the solution was concentrated by rotary evaporation under reduced pressure to give the glycan hydrolysate, which was directly subjected to ¹H-NMR and LC-MS measurements without further purification. This reaction protocol is applicable to hydrolysis of other glycans.

### 1.6 MALDI-TOF-MS

The stock solutions of saccharides (1.2×10⁻³ to 5×10⁻³ M) were prepared in dd-H₂O containing 0.1% formic acid and 50% CH₃CN. The stock solutions of matrix 2,5-DHB (10 mg/mL, 6.5×10⁻² M) and NaCl (1.7×10⁻² M) were prepared in dd-H₂O containing 0.1% formic acid/CH₃CN (1:1 v/v). The sample for MALDI-MS measurement was typically prepared by combining 10 μL of saccharide stock solution with 10 μL of matrix stock solution and 5 μL of NaCl solution to give a final volume of 25 μL in an Eppendorf tube. Then, 2 μL of this sample solution was applied to the sample plate by a dried-droplet method (i.e., placing a droplet of the sample solution on a mass spectrometer's sample stage and drying the droplet at room temperature),²⁴ instead of a vacuum drying process. Samples of saccharide-NAIM derivatives were similarly prepared for MALDI-MS determination. The mass spectrometer used to acquire the spectra was a Voyager Elite Applied Biosystems (Foster City, Calif., US). The accelerating voltage was set at 20 kV in either positive or negative ion mode. Typically, spectra were obtained by accumulating 800-1000 laser shots for quantification. Laser energy per pulse was calibrated with a laser power meter (PEM 101, Laser Technik, Berlin, Germany) so that laser fluence could be precisely measured. The delay extraction time was adjusted from 10 to 500 ns. The grid voltage was set at 95% of the accelerating voltage; the guide wire voltage was 0.2% of the accelerating voltage. The laser beam diameter was measured as ˜100 μm on the sample target. The laser fluence was in the range of 50-300 mJ/cm². The flight tube pressure inside the vacuum was always kept between 10⁻⁷ and 10⁻⁶ torr.

### 1.7 LC-MS

Velos Pro dual-pressure linear ion trap MS from Thermo Fisher Scientific (San Jose, Calif., US) was used for linear trap quadrupole Fourier transform mass spectrometry (LTQ-FTMS). The saccharide sample was similarly prepared as described above and subjected to LC-MS analysis. In brief, a sample solution was prepared by dissolving the saccharide (or sugar-NAIM derivative) in dd-H₂O (0.5 mL) containing 0.1% formic acid. The sample solution (5 μL) was then injected into a Xbridge C18 column (1.0 mm i.d.×15.0 cm, 3.5 μm particle size, and 130 Å pore size). The flow rate was set 0.05 mL/min, the gradient elution was applied (0-20 min, 2-98% CAN/H₂O) and a UV detector was used for the LTQ-FTMS analysis.

### 1.8 NMR

¹H-NMR spectra were recorded on a Bruker AV600 MHz NMR spectrometer (GmbH, Rheinstetten, Germany). This is a two-channel system equipped with a 5 mm DCI dual cryoprobe for high sensitivity ¹H/¹³C observation. The sugar-NAIM sample was dissolved in D₂O solution containing (CH₃)₂SO (0.03-0.1%) as an internal standard. Quantification of sugars was based on the integral areas of the characteristic proton signals. For example, the area of H-2 in individual hexose-NAIM derivative was compared with that of (CH₃)₂SO (integral region from δ 2.792 to 2.727 ppm for six protons of the two methyl groups). The acquisition parameters were equipped with a high-performance actively shielded standard bore 14.09 Tesla superconducting magnet. ¹H-NMR acquisition parameters: 90° pulse, P1=9.95 μs, PL1=−0.8 dB; relaxation delay D1=2 sec; number of acquisition aq=1.9530824 (s); type of baseline correction: quad; window function: EM; LB=0.5 Hz; software for spectral processing and regression analysis: TopSpin 3.0.

### 2. Results

### 2.1 Preparation of Sugar-NAIM Derivatives in a Flow Chemistry System

We have previously prepared a series of sugar-NAIM derivatives in batch-wise manner by treating aldoses with NADA and iodine in a flask with magnetic stirring.²² This reaction usually completed in 3-6 h at room temperature. The reaction time was reduced to 1-2 h on using a NAIM labeling kit through the enhanced concentration of NADA and iodine.²⁵ Using a flow chemistry system further improved the labeling reaction (FIG. 2). In a typical procedure, a solution of NADA (30.0 mg, 0.18 mmol) in HOAc (glacial, 3.0 mL), a solution of monosaccharide (15.0 mg, 0.08 mmol) in HOAc (3.0 mL) and a solution of iodine (3.8 mg, 0.03 mmol) in HOAc (3.0 mL) were mixed and reacted in a flow system over a period of 10 min at 60° C. (the reading of instrument setting) at a flow rate of 1 mL/min. The desired sugar-NAIM products were obtained and concentrated under reduced pressure to remove HOAc. The product was analyzed by ¹H-NMR, MALDI-TOF-MS and LC-MS without further purification.

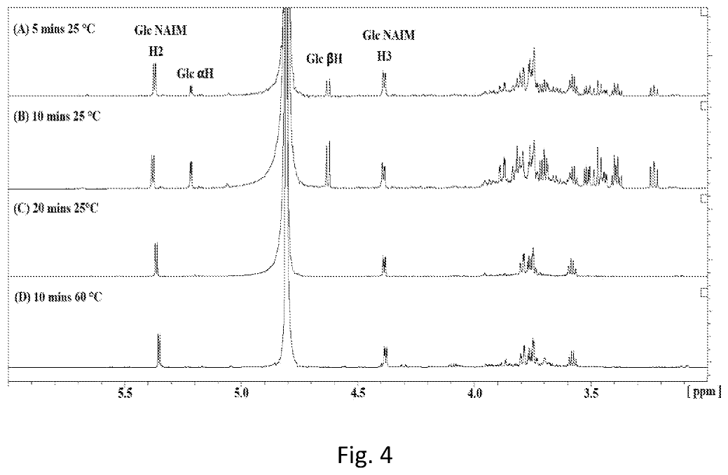

Taking D-glucose as an example, the formation of Glc-NAIM derivative was ˜20% for 5 min at 25° C. in a flow system, and essentially completed at 20 min (FIG. 4). The reaction time was reduced at 60° C. 10 min to give an essentially completed reaction. The reaction was monitored by ¹H-NMR spectra (600 MHz, D₂O). Glucose initially showed the C-1 proton signals at δ 5.22 and 4.64 for the α- and β-anomers, respectively. Both anomers were converted to a single NAIM compound, which displayed the characteristic C-2 and C-3 protons at δ 5.38 and 4.39, respectively.

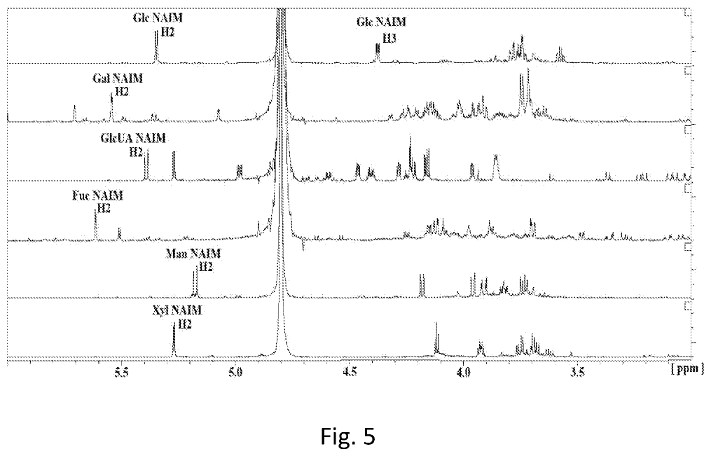

FIG. 5 shows that various monosaccharides including D-Glc, D-Gal, D-GlcUA, L-Fuc, D-Man and D-Xyl were effectively transformed into their corresponding NAIM derivatives by mixing with NADA and iodine at 60° C. for 10 min in a flow system. This protocol of flow chemistry was applicable to prepare the NAIM derivatives of oligosaccharides and higher glycans, albeit requiring a somewhat longer reaction time (˜20 min).

### 2.2 Glycan Hydrolysis in a Flow Chemistry System

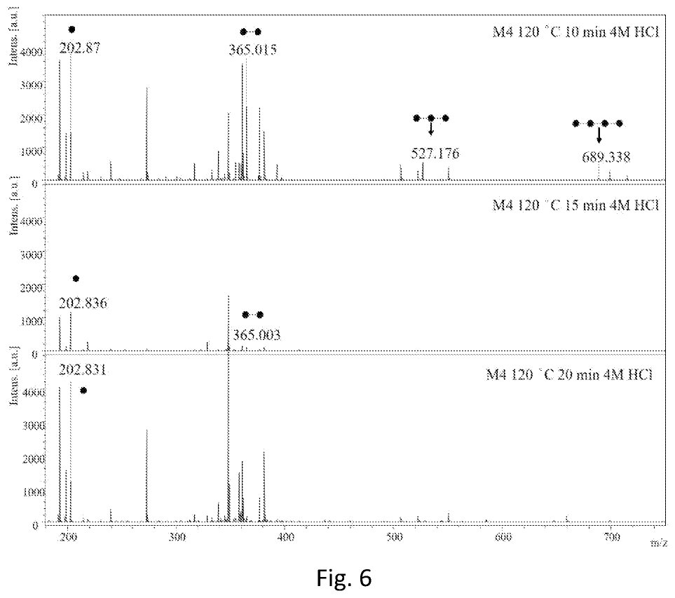

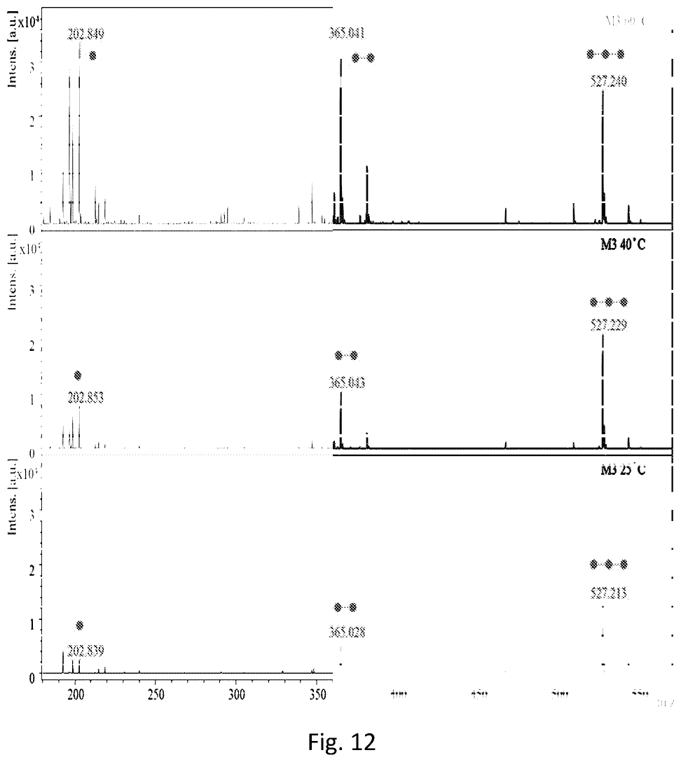

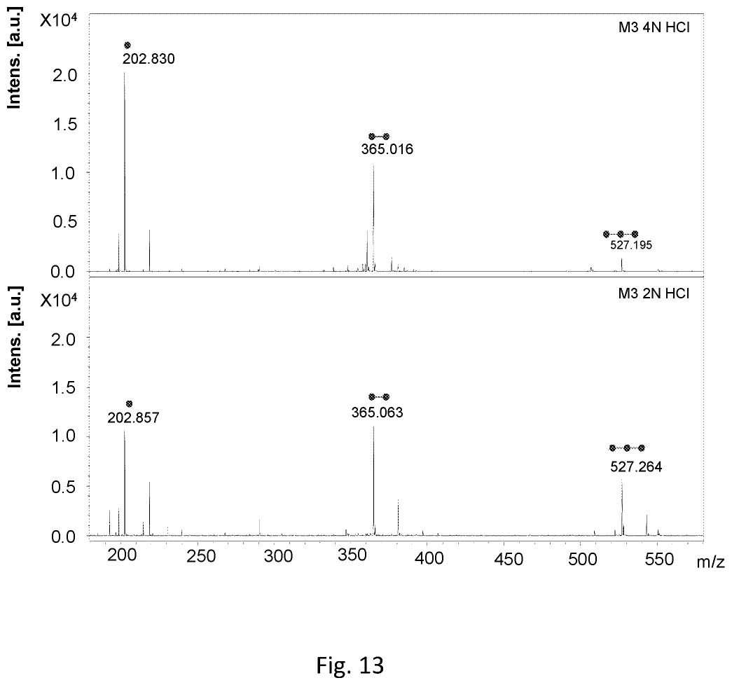

We first investigated the acidic hydrolysis of di-, tri- and tetrasaccharides in a flow chemistry system. Maltose (1.0 mg/mL) was treated with 4 M HCl at 80° C. for 10 min in a flow system to cause partial hydrolysis (˜65%) according to the MALDI-TOF-MS analysis of the product mixture (FIG. 11). The hydrolysis was accelerated at higher temperature (120 and 150° C.) and completed in 10 min. The temperature effect was further supported by the acidic hydrolysis of maltotriose (FIG. 12). On treatment of maltotriose with 4 M HCl at 25° C. for 10 min in a flow system, 15% of glucose and 30% of maltose were obtained while 55% of maltotriose remained. The rate of hydrolysis increased as the reaction temperature increased. After the acid treatment at 120° C. for 10 min, 95% of maltotriose was hydrolyzed to give 65% glucose and 30% maltose. Compared with FIG. 11, it seemed that the saccharide of higher size will slow down the hydrolysis rate. FIG. 13 compared the hydrolysis efficiency of maltotriose on treatment with 4 M or 2 M HCl at 120° C. for 10 min in a flow system. The hydrolysis of maltotriose apparently decreased in lower concentration of HCl. Thus, the hydrolysis of higher oligosaccharide, such as maltotetraose, was best conducted with 4 M HCl at 120° C. (FIG. 6). After 10 min reaction time, a mixture of maltotetraose (5%), maltotriose (15%), maltose (50%) and glucose (30%) was obtained according to the MALDI-TOF-MS analysis. Further degradation occurred after longer hydrolysis time (15 and 20 min); only glucose and maltose were observed as the sodiated ions at m/z 202 and 365, respectively.

We then investigated the degradation of a disaccharide that contained two different monosaccharide components in the flow chemistry system. Common saccharides (e.g., Glc, Man and Gal) are hardly distinguished by MS when they have the same molecular weight. High performance anion exchange chromatography with pulsed amperometric detection (HPAEC-PAD) is often used for direct separation and detection of the saccharide components by elution with strong base (NaOH).³³⁻³⁶ In comparison, the conventional reversed-phase high-pressure liquid chromatograph (HPLC) is more easily accessed to separate the proper derivatives of sugar components, such as sugar-NAIM compounds.^{22,37} In addition, HPLC can be linked with MS for analysis of oligosaccharides with prior derivatization.^{37,38} For glycan compositional analysis, those monosaccharides obtained from glycan hydrolysis were recycled into the flow system to generate the sugar-NAIM derivatives, even at low sample loads. The prepared sugar-NAIM derivatives were concentrated by rotary evaporation under reduced pressure, and analyzed by LC-MS without further purification.

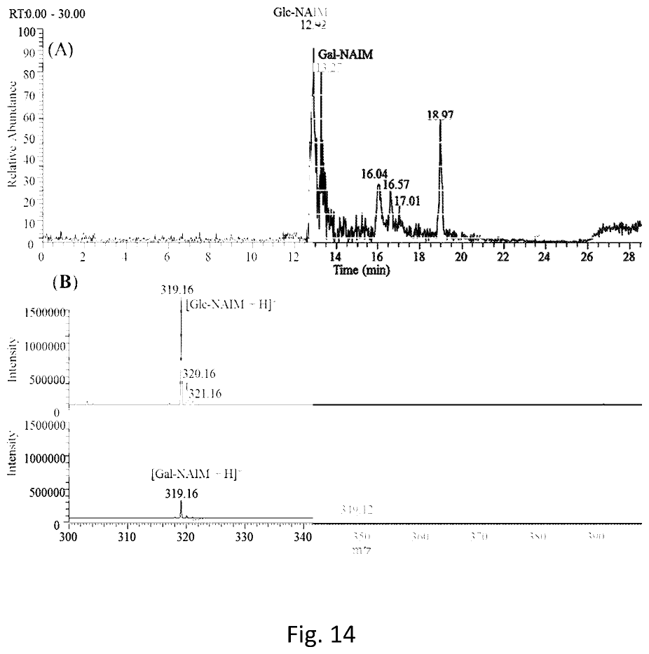

Taking lactose as an example, the glucose and galactose components were obtained by hydrolysis in a flow chemistry system. After NADA labeling, the Glc-NAIM and Gal-NAIM derivatives were analyzed by LC-MS. The residue was separable on a C18 capillary column and identified by linear trap quadrupole Fourier transform mass spectrometry (LTQ-FTMS) (FIG. 14). By tagging the NAIM chromophore, Glc-NAIM and Gal-NAIM occurring at the retention times of 13.3 min and 13.9 min were easily detected using a UV detector at a wavelength of 330 nm. The NAIM derivatives exhibited higher hydrophobicity than their parent saccharides to show enhanced MS signals.^{24,26} The isobaric isomers Glc-NAIM and Gal-NAIM both showed the protonated ions at m/z 319.

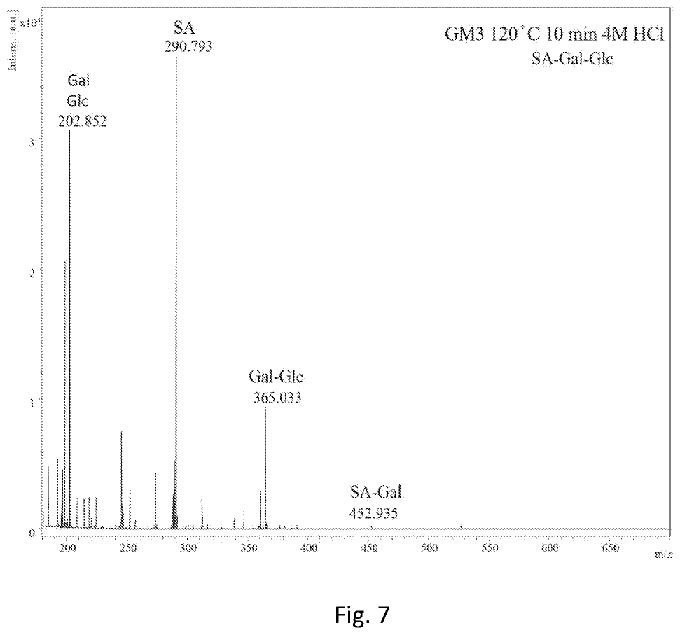

GM3 is a common glycosphingolipid in tissues. The carbohydrate portion (GM3-sugar) is a trisaccharide SA(2α,3)Gal(1β,4)Glc comprising sialic acid, galactose and glucose. In this study, GM3-sugar (5.0 mg, 8.0 μmol) was hydrolyzed with 4 M HCl at 120° C. for 10 min in a flow system, and the hydrolysate was analyzed by MALDI-TOF-MS (FIG. 7). Glc and Gal showed the sodiated molecular ion at m/z 203, whereas the signal at m/z 291 was attributable to SA with elimination of one water molecule. In comparison, the signal at m/z 365 ascribed to the Gal-Glc disaccharide (as the sodiated ion) was much stronger than the signal at m/z 453 attributable to the SA-Gal disaccharide (as the dehydrated ion). This result demonstrated that the sialyl glycoside bond was more susceptible to acid treatment, as expected.

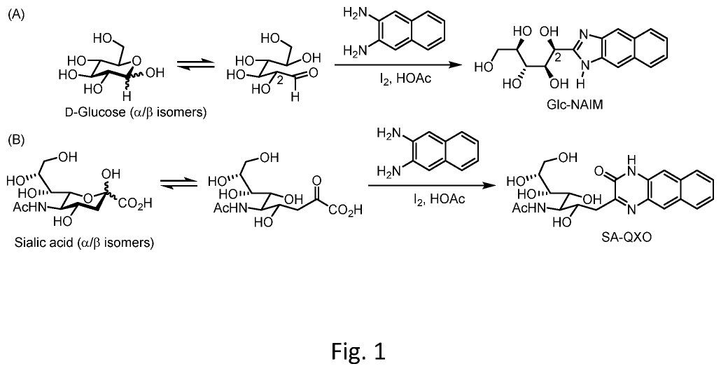

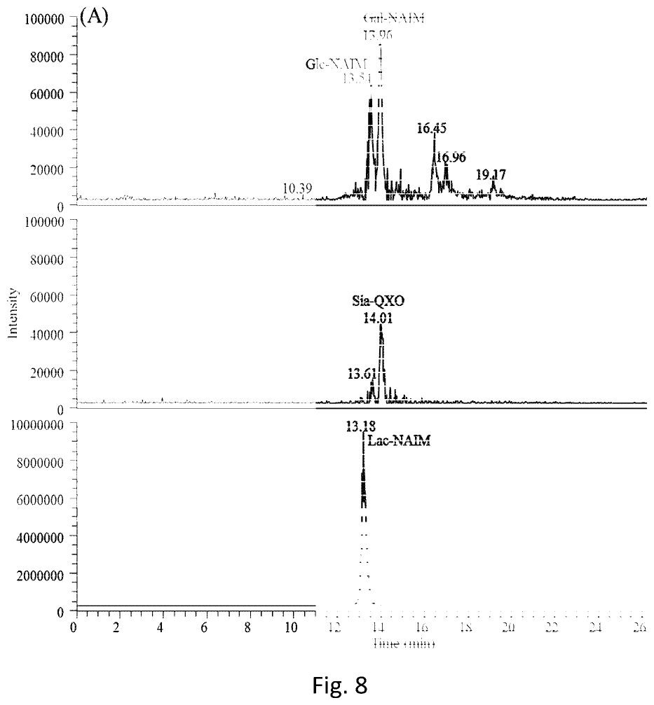

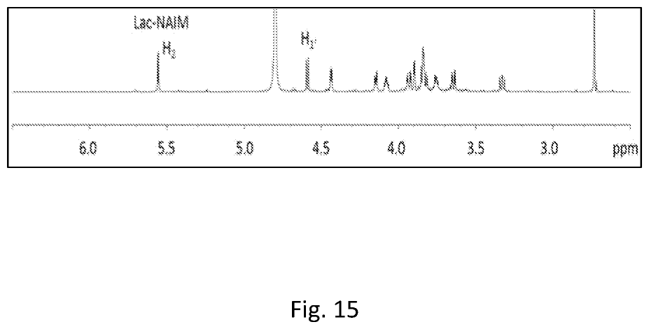

In addition to composition analysis, the lysate of GM3-sugar was concentrated and labeled with NADA in a flow chemistry system to obtain the corresponding NAIM and QXO derivatives (FIG. 1). The LC-MS analysis revealed four species of Glc-NAIM, Gal-NAIM, Lac-NAIM and SA-QXO occurring at 13.54, 13.96, 13.18 and 14.01 min, respectively (FIG. 8). Glc-NAIM, Gal-NAIM and Lac-NAIM displayed the [M+H]⁺ ions at m/z 319, 319 and 481, respectively, whereas SA-QXO exhibited the [M−H]⁻ ion at m/z 430. Of the most importance, Lac-NAIM (i.e., Gal-Glc-NAIM), but not Glc-Gal-NAIM, was identified by comparison with the retention time of authentic sample in the LC diagram (FIG. 8A), and the structure was confirmed by the ¹H-NMR spectroscopic determination (FIG. 15). Taking together, the results shown in FIG. 7 and FIG. 8 lead to the conclusion that the Glc moiety is at the reducing end, the SA moiety is at the non-reducing end, and the two moieties are linked with Gal to form the SA-Gal-Glc trisaccharide. This study thus provides an example of carbohydrate sequencing.

### 2.3 Developing a Continuous Protocol for Glycan Hydrolysis and Tandem NADA Tagging in a Flow Chemistry System

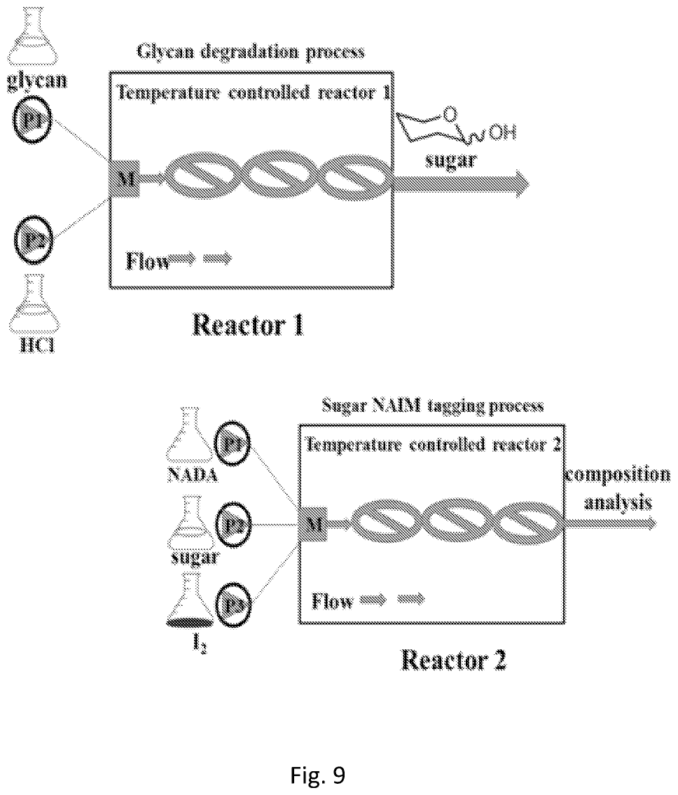

We further combined glycan hydrolysis and NADA tagging in a continuous flow system to simplify the procedure for preparation of sugar NAIM (or QXO) derivatives. An additional peristaltic pump reactor was installed to the Vapourtec E-series Flow Chemistry System (FIG. 9). For example, lactose (2.0 mg, 5.8 μmol) was suspended in HOAc (glacial, 2.0 mL) containing a small amount (2 μL) of 12 M HCl, and pumped into reactor-1 for hydrolysis. The reaction was performed at 120° C. for 15 min, and the glycan hydrolysate was pumped into reactor-2 for NADA tagging in HOAc solution at 60° C. for 10 min. The final mixture was concentrated by rotary evaporation under reduced pressure to give the sugar-NAIM derivatives, which were directly analyzed by MS and NMR to determine the composition of the glycan precursor.

### 2.4 Prospect for Glycan Structural Analysis

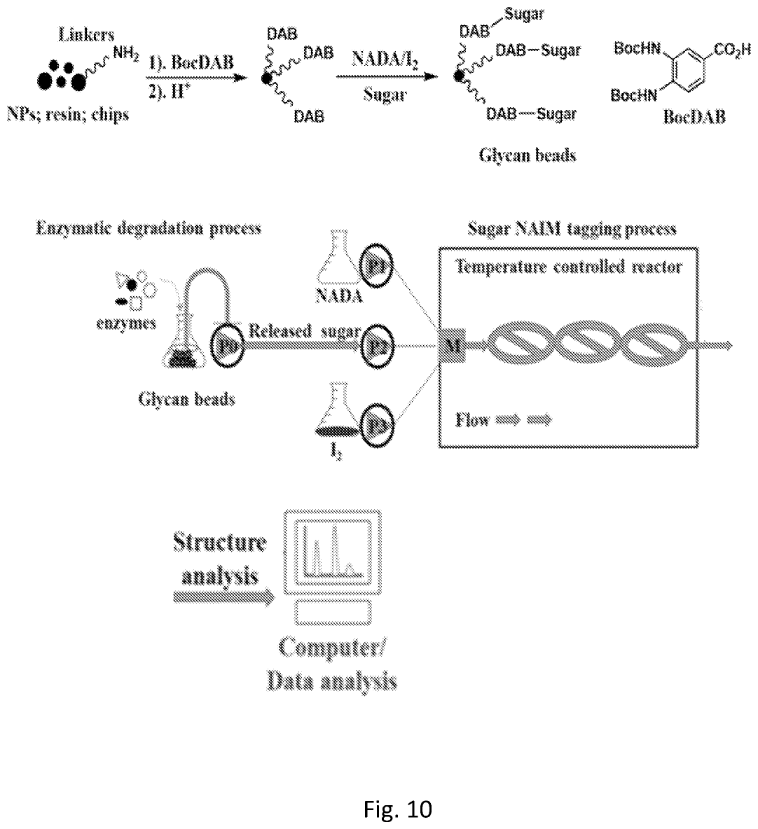

Automated polymer-supported synthesis of oligosaccharides is rapidly progressing.³⁹⁻⁴¹ Immobilization of complex glycan onto a polymer or solid surfaces can be advanced to structural analysis with the assistance of flow chemistry system. We have previously demonstrated that an arginine-tagged phenylenediamine can successfully catch tetrasialic acid.²⁴ We thus propose to modify the surface of polymer (or solid) with ortho-phenylenediamine moieties as depicted in FIG. 10. Many polymeric and solid materials carrying linkers with terminal amine groups are either commercially available or readily prepared.⁴² For example, porous silica beads are treated with 3-aminopropyltriethoxysilane to graft amino functional groups onto their surfaces. The solid support carrying linkers with terminal amine groups will be modified with the tert-butoxycarbonyl (Boc) protected 3,4-diaminobenzoic acid (DAB) via amide bond formation.²⁴ Then, the DAB encapsulated solid support can be used to catch the target glycan via the condensation reaction with its terminal aldehyde (or ketoacid) group at the reducing end.

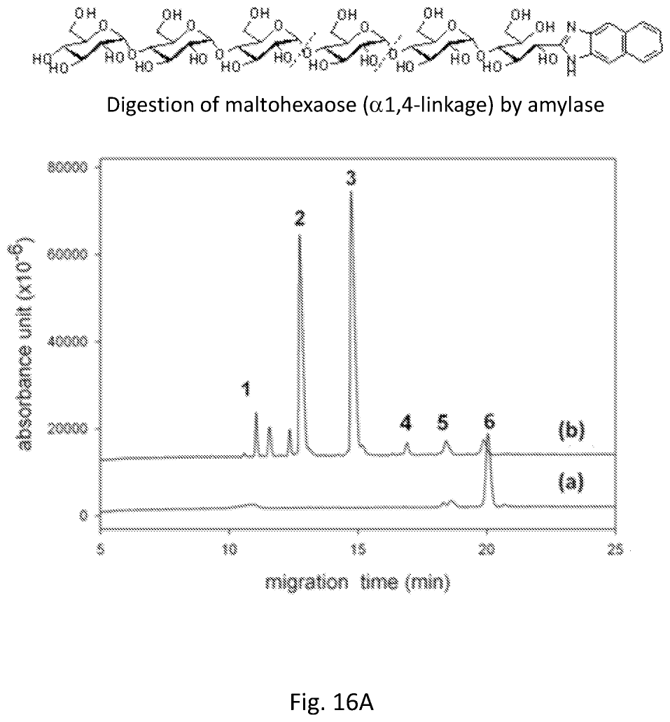

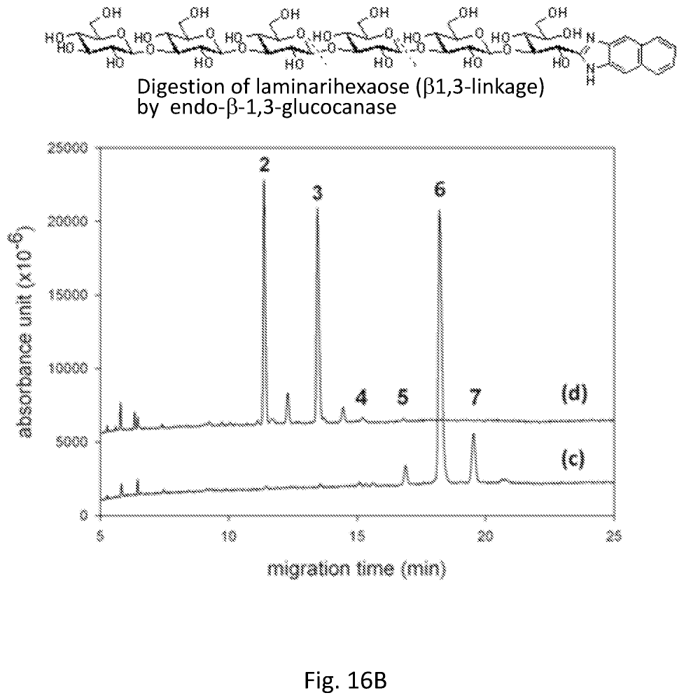

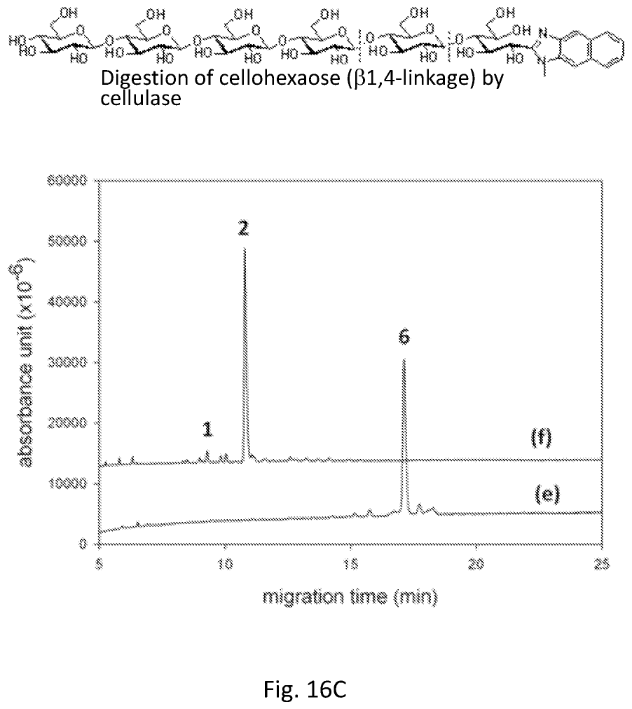

We have previously demonstrated the use of α-amylase, endo-β-1,3-glucanase and cellulose for specific digestion of maltohexaose, laminarihexaose and cellohexaose, respectively (FIGS. 16A-16C).⁴³ Siuzdak and coworkers have also devised an on-chip enzymatic reactions of galactosidase and sialyltransferase.⁴⁴ Therefore, enzymatic digestion (or acidic hydrolysis) of glycan on beads will be feasible to release saccharide components. Different glycosidases can be used to digest specific type of glycans,^{9,42} and the degree of glycoside bond cleavage can be controlled by the reaction conditions. The glycan hydrolysate can be subjected to NADA labeling in a flow system to obtain the corresponding sugar-NAIM (or sugar-QXO) derivatives for compositional analysis. This procedure is also possibly applicable to sequencing the glycan that contains heterosugars.

### 3. Conclusion

In this study, we demonstrated that glycan hydrolysis and saccharide tagging were accelerated in a flow chemistry system. Aldoses and α-ketoacid type saccharide components were mixed with NADA and iodine at 60° C. for 10 min to form the light-absorbing sugar-NAIM and sugar-QXO derivatives. This new method improved the structural determination, compositional analysis and possibly sequencing of the parent glycan by using a combination of LC, MS and NMR techniques. For example, the hetero trisaccharide GM3-sugar was hydrolyzed in 4 M HCl at 120° C. for 10 min and NADA labeled in a flow system. Since the product mixture was found to contain Glc-NAIM, Gal-NAIM, Lac-NAIM and SA-QXO by MALDI-TOF-MS, LC-MS and ¹H-NMR analyses, the results concluded that GM3-sugar has a sequence of SA-Gal-Glc out of 6 possible permutations. As demonstrated in this study, application of flow chemistry system for continuous glycan hydrolysis and NADA labeling can assist the existing methods in glycan sequencing. At this moment, we still use micromolar amount of glycan sample; however, one should be able to conduct this experimental protocol with smaller amounts of glycans when advanced instruments are available. For complete glycan sequencing, one must elucidate the linkage position and anomeric configuration in each monosaccharide component. This is still a challenging task, even though many hurdles have been overcome by using chemical, biological and instrumental methods in concert.^{2,3,9,17}

### REFERENCES

1. Varki, A. Biological Roles of Glycans. Glycobiology 2017, 27, 3-49.
2. Levery, S. B.; Hakomori, S. Micro-scale Methylation Analysis of Glycolipids Using Capillary Gas Chromatography-Chemical Ionization Mass Fragmentography with Selected Ion Monitoring. Methods Enzymol. 1987, 138, 13-25.
3. Welply, J. K. Sequencing Methods for Carbohydrates and Their Biological Applications. Trends Biotechnol. 1989, 7, 5-10.
4. Leymarie, N.; Zaia, J. Effective Use of Mass Spectrometry for Glycan and Glycopeptide Structural Analysis. Anal. Chem. 2012, 84, 3040-3048.
5. Pabst, M.; Altmann, F. Glycan Analysis by Modern Instrumental Methods. Proteomics 2011, 11, 631-643.
6. Fagerson, I. S. Thermal Degradation of Carbohydrates. J. Arg. Food Chem. 1969, 17, 747-750.
7. Thompson, D. R.; Grethlein, H. E. Design and Evaluation of a Plug Flow Reactor for Acid Hydrolysis of Cellulose. Ind. Eng. Chem. Prod. Res. Dev. 1979, 18, 166-169.
8. Fan, J.-Q.; Namiki, Y.; Matsuoka, K.; et al. Comparison of Acid Hydrolytic Conditions for Asn-linked Oligosaccharides. Anal. Biochem. 1994, 219, 375-378.
9. Prime, S.; Dearnley, J.; Ventom, A. M.; et al. Oligosaccharide Sequencing Based on Exo- and Endoglycosidase Digestion and Liquid Chromatographic Analysis of the Products. J. Chromat. A 1996, 720, 263-274.
10. Zhao, X.; Zhou, Y.; Liu, D. Kinetic Model for Glycan Hydrolysis and Formation of Monosaccharides during Dilute Acid Hydrolysis of Sugarcane Bagasse. Bioresour. Technol. 2012, 105, 160-168.
11. Song, X.; Ju, H.; Lasanajak, Y.; et al. Oxidative Release of Natural Glycans for Functional Glycomics. Nat. Methods 2016, 13, 528-536.
12. Ruhaak, L. R.; Zauner, G.; Huhn, C.; et al. Glycan Labeling Strategies and Their Use in Identification and Quantification. Anal. Bioanal. Chem. 2010, 397, 3457-3481.
13. Harvey, D. J. Derivatization of Carbohydrates for Analysis by Chromatography, Electrophoresis and Mass spectrometry. J. Chromatogr. B 2011, 879, 1196-1225.
14. Gennaro, L. A.; Delaney, J.; Vouros, P.; et al. Capillary Electrophoresis/Electrospray Ion Trap Mass Spectrometry for the Analysis of Negatively Charged Derivatized and Underivatized Glycans. Rapid Commun. Mass Spectrom. 2002, 16, 192-200.
15. Okatch, H.; Torto, N. Profiling of Carbohydrate Polymers in Biotechnology Using Microdialysis Sampling, High Performance Anion Exchange Chromatography with Integrated Pulsed Electrochemical Detection/Mass Spectrometry. Afric. J. Biotechnol. 2003, 2, 764-778.
16. Zhong, X.; Chen, Z.; Snovida, S.; et al. Capillary Electrophoresis-Electrospray Ionization-Mass Spectrometry for Quantitative Analysis of Glycans Labeled with Multiplex Carbonyl-reactive Tandem Mass Tags. Anal. Chem. 2015, 87, 6527-6534.
17. Ashline, D.; Singh, S.; Hanneman, A.; et al. Congruent Strategies for Carbohydrate Sequencing. 1. Mining Structural Details by MSⁿ. Anal. Chem. 2005, 77, 6250-6262.
18. Pompach, P.; Chandler, K. B.; Lan, R.; et al. Semi-automated Identification of N-Glycopeptides by Hydrophilic Interaction Chromatography, Nano-Reverse-Phase LC-MS/MS, and Glycan Database Search. J. Proteome Res. 2012, 11, 1728-1740.
19. Everest-Dass, A. V.; Abrahams, J. L.; Kolarich, D.; et al. Structural Feature Ions for Distinguishing N- and O-linked Glycan Isomers by LC-ESI-IT MS/MS. J. Am. Soc. Mass Spectrom. 2013, 24, 895-906.
20. Barb, A. W.; Prestegard, J. H. NMR Analysis Demonstrates Immunoglobulin GN-glycans are Accessible and Dynamic. Nat. Chem. Biol. 2011, 7, 147-153.
21. Bøjstrup, M.; Petersen, B. O.; Beeren, S. R.; et al. Fast and Accurate Quantitation of Glycans in Complex Mixtures by Optimized Heteronuclear NMR Spectroscopy. Anal. Chem. 2013, 85, 8802-8808.
22. Lin, C.; Lai, P.-T.; Liao, K.-S.; et al. Using Molecular Iodine in Direct Oxidative Condensation of Aldoses with Diamines: An Improved Synthesis of Aldo-benzimidazoles and Aldo-naphthimidazoles for Carbohydrate Analysis. J. Org. Chem. 2008, 73, 3848-3853.
23. Lin, C.; Hung, W.-T.; Kuo, C.-Y.; et al. I₂-catalyzed Oxidative Condensation of Aldoses with Diamines: Synthesis of Aldo-naphthimidazoles for Carbohydrate Analysis. Molecules 2010, 15, 1340-1353.
24. Chang, Y.-L.; Liao, K.-S.; Chen, Y.-C.; et al. Tagging Saccharides for Signal Enhancement in Mass Spectrometric Analysis. J. Mass Spectrom. 2011, 46, 247-255.
25. Chen, Y.-T.; Wang, S.-H.; Hung, W.-T.; et al. Quantitative Analysis of Sugar Ingredients in Beverages and Food Crops by an Effective Method Combining Naphthimidazole Derivatization and ¹H-NMR Spectrometry. Func. Food Health Dis. 2017, 7, 494-510.
26. Lin, C.; Hung, W.-T.; Chen, C.-H.; et al. A New Naphthimidazole Derivative for Saccharide Labeling with Enhanced Sensitivity in Mass Spectrometry Detection. Rapid Commun. Mass Spectrom. 2010, 24, 85-94.
27. Lin, C.; Kuo, C. Y.; Liao, K. S.; et al. Monosaccharide-NAIM Derivatives for D-, L-Configuration Analysis. Molecules 2011, 16, 652-664.
28. Chen, Y.-T.; Chen, K.-H.; Fang, W.-F.; et al. Flash Synthesis of Carbohydrate Derivatives in Chaotic Microreactors. Chem. Eng. J. 2011, 174, 421-424.
29. Kumar, S.; Gupta, R. B. Hydrolysis of Microcrystalline Cellulose in Subcritical and Supercritical Water in a Continuous Flow Reactor. Ind. Eng. Chem. Res. 2008, 47, 9321-9329.
30. Wegner, J.; Ceylan, S.; Kirschning, A. Ten Key Issues in Modern Flow Chemistry. Chem. Commun. 2011, 47, 4583-4592.
31. Pastre, J. C.; Browne, D. L.; Ley, S. V. Flow Chemistry Syntheses of Natural Products. Chem. Soc. Rev. 2013, 42, 8849-8869.
32. Hessel, V.; Kralisch, D.; Kockmann, N.; et al. Novel Process Windows for Enabling, Accelerating, and Uplifting Flow Chemistry. ChemSusChem. 2013, 6, 746-789.
33. Hanko, V. P.; Rohrer, J. S. Determination of Carbohydrates, Sugar Alcohols, and Glycols in Cell Cultures and Fermentation Broths Using High-Performance Anion-Exchange Chromatography with Pulsed Amperometric Detection. Anal. Biochem. 2000, 283, 192-199.
34. Hardy, M. R.; Townsend, R. R. Separation of Positional Isomers of Oligosaccharides and Glycopeptides by High-Performance Anion-Exchange Chromatography with Pulsed Amperometric Detection. Proc. Natl. Acad. Sci. USA 1988, 85, 3289-3293.
35. Townsend, R. R.; Hardy, M. R.; Hindsgaul, O.; et al. High-Performance Anion-Exchange Chromatography of Oligosaccharides Using Pellicular Resins and Pulsed Amperometric Detection. Anal. Biochem. 1988, 174, 459-470.
36. Townsend, R. R.; Hardy, M.; Olechno, J. D.; et al. Chromatography of Carbohydrates. Nature 1988, 335, 379-380.
37. Broberg, A. High-Performance Liquid Chromatography/Electrospray Ionization Ion-trap Mass Spectrometry for Analysis of Oligosaccharides Derivatized by Reductive Amination and N,N-Dimethylation. Carbohydr. Res. 2007, 342, 1462-1469.
38. Hung, W.-T.; Wang, S.-H.; Chen, C.-H.; et al. Tagging N-Linked Glycan with 2,3-Naphthalenediamine for Mass Spectrometric Analysis. J. Chin. Chem. Soc. 2013, 60, 955-960.
39. Haase, W. C.; Seeberger, P. H. Recent Progress in Polymer-Supported Synthesis of Oligosaccharides and Carbohydrate Libraries. Curr. Org. Chem. 2000, 4, 481-511.
40. Belog, G.; Zhu, T.; Boons, G. J. Polymer-Supported Oligosaccharide Synthesis by a Loading-Release-Reloading Strategy. Tetrahedron Lett. 2000, 41, 6969-6972.
41. Seeberger, P. H.; Werz, D. B. Synthesis and Medical Applications of Oligosaccharides. Nature 2007, 446, 1046-1051.
42. Solid-Phase Synthesis: A Practical Guide. Kates, S. A.; Albericio, F. (Eds). CRC Press, 2000, Marcel Dekker Inc., New York.
43. Kuo, C.-Y.; Wang, S.-H.; Lin, C.; et al. Application of 2,3-Naphthalenediamine in Labeling Natural Carbohydrates for Capillary Electrophoresis. Molecules 2012, 17, 7387-7400.
44. Northen, T. R.; Lee, J. C.; Hoang, L.; et al. A Nanostructure-Initiator Mass Spectrometry-based Enzyme Activity Assay. Proc. Natl. Acad. Sci. USA 2008, 105, 3678.

What is claimed is:

1. A method for carbohydrate analysis, comprising the steps of:
(i) degrading a glycan molecule in a hydrolysis reaction to produce a glycan hydrolysate containing monosaccharides;
(ii) labeling the monosaccharides with a detectable label in a sugar derivatization reaction to produce sugar derivatives;
(iii) analyzing the sugar derivatives for measurement of one or more characteristics of the sugar derivatives; and
(iv) determining the composition and/or structure of the glycan molecule based on the one or more characteristics of the sugar derivatives,
wherein the glycan hydrolysis reaction of step (i) and the sugar derivation reaction of step (ii) are performed in a flow chemistry system.

2. The method of claim 1, wherein the glucan molecule comprises oligosaccharides (e.g. di, tri, tetra saccharides) and/or polysaccharides.

3. The method of claim 1, wherein the fluorescent label is a naphthimidazole molecule.

4. The method of claim 1, wherein the sugar derivatives are analyzed by nuclear magnetic resonance spectroscopy (NMR), liquid chromatography (LC), gas chromatography (GC), mass spectrometry (MS) and any combination thereof.

5. The method of claim 1, wherein the glycan hydrolysis reaction is performed in a hydrolysis unit, the sugar derivation reaction is performed in a derivatization unit, and the hydrolysis unit is connected to the derivatization unit via connective tubing to provide a continuous flow path where the glycan hydrolysate flows from the hydrolysis unit into the derivatization unit for the sugar derivatization reaction.

6. The method of claim 1, wherein the monosaccharides are selected from the group consisting of ribose (Rib), arabinose (Ara), xylose (Xyl), rhamnose (Rha), fucose (Fuc), glucose (Glc), mannose (Man), galactose (Gal), N-acetylgalactosamine (GalNAc), glucuronic acid (GlcUA), galacturonic acid (GalUA) and any combination thereof.

7. The method of claim 1, wherein the hydrolysis reaction is effected by acidic hydrolysis or enzymatic hydrolysis.

8. The method of claim 7, wherein the acidic hydrolysis is carried out under pH 1-5 at a temperature in the range of 60° C. to 150° C. for 5 to 120 minutes.

9. The method of claim 7, wherein the enzymatic hydrolysis is performed with one or more enzymes selected from the group consisting of amylase, glucanase, cellulase, galactosidase, neuraminidase, glycosyltransferase, sialyltransferase, and any combinations thereof.

10. A flow chemistry system for carbohydrate analysis, comprising
(i) a hydrolysis unit for performing a hydrolysis reaction to degrade a glycan molecule to produce a glycan hydrolysate containing monosaccharides; and
(ii) a derivatization unit for performing a sugar derivatization reaction to label the monosaccharides with a detectable label to produce d sugar derivatives,
wherein the hydrolysis unit is connected to the derivatization unit via connective tubing to provide a continuous flow path where the glycan hydrolysate flows from the hydrolysis unit into the derivatization unit for the sugar derivatization reaction.

11. An apparatus for carbohydrate analysis, which comprises
(a) a flow chemistry system, comprising
(i) a hydrolysis unit for performing a hydrolysis reaction to degrade a glycan molecule to produce a glycan hydrolysate containing monosaccharides; and
(ii) a derivatization unit for performing a sugar derivatization reaction to label the monosaccharides with a detectable label to produce sugar derivatives,
wherein the hydrolysis unit is connected to the derivatization unit via connective tubing to provide a continuous flow path where the glycan hydrolysate flows from the hydrolysis unit into the derivatization unit for the sugar derivatization reaction;
(b) an analytical system adapted for interaction with the flow chemistry system for the measurement of one or more characteristics of the sugar derivatives;
(c) a data processing system comprising sugar database and a means for comparing the one or more characteristics of the sugar derivatives measured by the analytical system with the sugar database to determine the composition and sugar sequence of the glycan molecule.

12. The system of claim 11, wherein
the hydrolysis unit includes a first reservoir A containing a solution of the glycan molecule, a first reservoir B containing an acidic solution, a first reactor and a first collection valve, connected with connective tubing and configured to enable the solution of the glycan molecule and the acidic solution to flow into the first reactor where the hydrolysis reaction is performed and the resultant glycan hydrolysate flows into the derivatization unit when the collection valve is in an open position.

13. The system of claim 11, wherein
the derivatization unit includes a second reservoir A containing the fluorescent label, a second reservoir B containing the glycan hydrolysate, a mixer, a second reactor and a second collection valve, connected with connective tubing and configured to enable the label and the glycan hydrolysate to flow into the mixer to form a mixture of the label and the glycan hydrolysate, and the mixture to flow into the reactor to produce the sugar derivatives.

14. The system of claim 11, wherein the measurement is performed by nuclear magnetic resonance spectroscopy (NMR), liquid chromatography (LC), gas chromatography (GC), mass spectrometry (MS) and any combination thereof.
